(19) United States
(12) Patent Application Publication
(10) Pub. No.: US 2026/0272666 A1
(43) Pub. Date: Sep. 17, 2026

(54) WIRE ROPE ARTICULATING EXPANDABLE TLIF

(71) Applicant: GLOBUS MEDICAL, INC., AUDUBON, PA (US)

(72) Inventor: Shawn Cox, Shrewsbury, PA (US)

(21) Appl. No.: 19/079,565

(22) Filed: Mar. 14, 2025

(51) Int. Cl. A61F 2/44; A61F 2/30

(52) U.S. Cl. CPC A61F 2/442; A61F 2002/30593

(57) ABSTRACT

Disclosed herein are embodiments of a spacer for positioning between first and second bones of adjacent vertebral bodies, the spacer having a proximal end having a proximal end longitudinal axis and having a first endplate configured to engage the first bone, a second endplate configured to engage the second bone; and an expansion wedge positioned between the first endplate and second endplate. The spacer may further include a distal end connected to the proximal end and having a distal end longitudinal axis, a drive nut extending from the distal end, and a wire rope assembly extending down a center of the spacer from the proximal end to the distal end.

### FIELD OF THE INVENTION

The various embodiments disclosed herein relate generally to the stabilization of adjacent vertebrae of the spine by inserting an intervertebral spacer, and more particularly an intervertebral spacer that is adjustable in height.

### BACKGROUND

The vertebral or spinal column (spine, backbone) is a flexible assembly of vertebrae stacked on top of each other extending from the skull to the pelvic bone which acts to support the axial skeleton and to protect the spinal cord and nerves. The vertebrae are anatomically organized into four generalized body regions identified as cervical, thoracic, lumbar, and sacral; the cervical region including the top of the spine beginning in the skull, the thoracic region spanning the torso, the lumbar region spanning the lower back, and the sacral region including the base of the spine ending with connection to the pelvic bone. With the exception of the first two cervical vertebrae, cushion-like discs separate adjacent vertebrae, i.e. intervertebral discs.

The stability of the vertebral column during compression and movement is maintained by the intervertebral discs. Each disc includes a gel-like center surrounded by a fibrous ring. The gel-like center, i.e. nucleus pulposus, provides strength such that the disc can absorb and distribute external loads and contains a mixture of type II-collagen dispersed in a proteoglycan matrix. The fibrous ring, or annulus fibrosus, provides stability during motion and contains laminated rings of type-I collagen. Thus, the annulus fibrosis and the nucleus pulposus are interdependent, as the annulus fibrosis contains the nucleus pulposus in place and the nucleus pulposus aligns the annulus fibrosus to accept and distribute external loads. The integrity of the composition and structure of the intervertebral disc is necessary to maintain normal functioning of the intervertebral disc.

Many factors can adversely alter the composition and structure of the intervertebral disc, such as normal physiological aging, mechanical injury/trauma, and/or disease, resulting in impairment or loss of disc function. For example, the content of proteoglycan in the nucleus pulposus declines with age, thus, it follows that the ability of the nucleus pulposus to absorb water concurrently declines. Therefore, in normal aging the disc progressively dehydrates, resulting in a decrease in disc height and possible de-lamination of the annulus fibrosus.

Mechanical injury can tear the annulus fibrosis allowing the gel-like material of the nucleus pulposus to extrude into the spinal canal and compress neural elements. Growth of a spinal tumor can impinge upon the vertebrae and/or disc potentially compressing nerves.

Bones of the spine, and bony structures, generally, are susceptible to a variety of weaknesses that can affect their ability to provide support and structure. Weaknesses in bony structures have numerous potential causes, including degenerative diseases, tumors, fractures, and dislocations. Advances in medicine and engineering have provided doctors with a plurality of devices and techniques for alleviating or curing these weaknesses.

In some cases, the spinal column, in particular, requires additional support in order to address such weaknesses. One technique for providing support is to insert a spacer between adjacent vertebrae.

### SUMMARY

The embodiments herein provide a spacer for positioning between first and second bones of adjacent vertebral bodies, the spacer having a proximal end having a proximal end longitudinal axis and having a first endplate configured to engage the first bone, a second endplate configured to engage the second bone; and an expansion wedge positioned between the first endplate and second endplate. The spacer may further include a distal end connected to the proximal end and having a distal end longitudinal axis, a drive nut extending from the distal end, and a wire rope assembly extending down a center of the spacer from the proximal end to the distal end.

The embodiments herein also provide a spacer for positioning between first and second bones of adjacent vertebral bodies, the spacer having a proximal end containing a first endplate configured to engage the first bone, a second endplate configured to engage the second bone, and an expansion wedge positioned between the first endplate and second endplate.

Embodiments may also have a hinge connected to the proximal end, a distal end connected to the hinge, a drive nut extending from the hinge, an anchor terminal positioned inside the expansion wedge, a hollow threaded terminal positioned inside the drive nut, and a flexible member connecting between the anchor terminal and hollow threaded terminal.

The embodiments herein also provide a spacer for positioning between first and second bones of adjacent vertebral bodies, the spacer having a proximal end comprising a first endplate configured to be positioned between the first and second bones, a second endplate configured to be positioned between the first and second bones, and an expansion wedge positioned between the first endplate and second endplate. Embodiments of the spacer may also have a hinge connected to the proximal end, a sweeping void positioned inside the hinge, a distal end connected to the hinge, a drive nut extending from the hinge, and an anchor terminal positioned inside the expansion wedge. Some embodiments may also have a hollow threaded terminal positioned inside the drive nut and having interior female threads sized to engage with an inserter and exterior male threads sized to engage with female threads inside the drive nut, and a wire rope member connecting between the anchor terminal and hollow threaded terminal and permitted to bend within the sweeping void.

### BRIEF DESCRIPTION OF THE DRAWINGS

A more complete understanding of the disclosure, and the attendant advantages and features thereof, will be more readily understood by reference to the following detailed description when considered in conjunction with the accompanying drawings, in which:

FIG. 1 is a perspective view of an embodiment of an articulating and expandable spacer.

FIG. 2 is a perspective view of an embodiment of the spacer where the proximal end of the spacer has pivoted to an angle relative to the distal end of the spacer.

FIG. 3 is a perspective view of an embodiment of the spacer showing an initial step in the procedure where the spacer is being attached to an inserter.

FIG. 4 is a perspective view of an embodiment of the spacer where the spacer is being inserted into the body using the inserter.

FIG. 5 is a section view of an embodiment of the spacer taken vertically along the longitudinal axis of the distal end and the proximal end.

FIG. 6 is a perspective view of an embodiment of the wire rope assembly.

FIG. 7 is a perspective section view of an embodiment of the spacer taken along the longitudinal axis of the distal end and the proximal end.

FIG. 8 is a perspective view of a driver engaging with the drive nut to cause rotation of the drive nut.

FIG. 9 is a perspective view of a driver engaging with the drive nut to cause rotation of the drive nut where the driver has been hidden to show the translation of the hollow threaded terminal.

FIG. 10 is a section view of an embodiment of the spacer taken along the longitudinal axis of the distal end and the proximal end where the plates are in their initial state.

FIG. 11 is a section view of an embodiment of the spacer taken along the longitudinal axis of the distal end and the proximal end where the plates are expanding and the hollow threaded terminal is translating.

FIG. 12 is a section view of an embodiment of the spacer where the proximal end has articulated or pivoted relative to the distal end and the driver is engaged with the drive nut.

FIG. 13 is a section view of an embodiment of the spacer where the proximal end has articulated or pivoted relative to the distal end and the rotation of the drive nut is causing the terminal anchor and the hollow threaded terminal to translate along their respective longitudinal axes.

FIG. 14 is a perspective view of an embodiment of the spacer where the proximal end has articulated or pivoted relative to the distal end and the driver is engaged with the drive nut.

FIG. 15 is a section view of an embodiment of the spacer where the proximal end has articulated or pivoted relative to the distal end and the end plates have been expanded while the spacer is in this angled position.

### DETAILED DESCRIPTION OF THE DRAWINGS

As required, detailed embodiments are disclosed herein; however, it is to be understood that the disclosed embodiments are merely examples and that the systems and methods described below can be embodied in various forms. Therefore, specific structural and functional details disclosed herein are not to be interpreted as limiting, but merely as a basis for the claims and as a representative basis for teaching one skilled in the art to variously employ the present subject matter in virtually any appropriately detailed structure and function. Further, the terms and phrases used herein are not intended to be limiting, but rather, to provide an understandable description of the concepts.

The terms “a” or “an”, as used herein, are defined as one or more than one. The term plurality, as used herein, is defined as two or more than two. The term another, as used herein, is defined as at least a second or more. The terms “including” and “having,” as used herein, are defined as comprising (i.e., open language).

The Transforaminal Lumbar Interbody Fusion (TLIF) procedure is a posterior approach to interbody fusion that involves the removal of bony structures to directly decompress nerves, followed by the insertion of a device into the disc space at around 15-30° oblique angle from midline for additional indirect decompression and increased stability. There are a variety of interbody options for the TLIF procedure including static straight, expandable straight, static articulating, and expandable articulating. This approach can be done through either open or MIS techniques and requires supplemental fixation.

Articulating spacers are different than straight spacers because after they've been inserted into the disc space, the surgeon can steer them into a more anterior position, with the length of the spacer spanning medial-lateral. This positioning offers additional benefits. Due to the more anterior positioning, they can adequately restore the natural lordosis of the lumbar spine and additionally allow for a larger footprint on stronger apophyseal ring bone for a lot of surface area contact and a potentially lower subsidence rate. Articulating cages that also expand in height provide additional value by reducing the disruption to surrounding anatomy due to the low starting heights of the spacer. These devices also provide a better patient fit with continuous height expansion. This customized patient fit may also prevent post-op spacer migration which is potential complication following TLIFs.

One of the challenges with an expandable articulating TLIF is the mechanism by which it achieves change in height. Most straight expandable spacers operate via a drive screw and ramps, however the angled positioning of an articulating spacer complicates such a mechanism as the insertion trajectory is seldom aligned with the length of the spacer, which is typically where the drive screw would be located. Therefore, the exemplary embodiments herein provide a number of novel solutions for creating spacer height expansion at a variable angle.

The embodiments herein provide a spacer which is capable of articulation or hinging or pivoting once inserted into the desired joint. And following this articulation, while holding this position, the drive nut can still be rotated thus causing the plates to expand, increasing the width of the spacer. This allows embodiments of the spacer to be inserted into the body at a first angle, pivot a proximal end of the spacer about a hinge, and then expand the spacer.

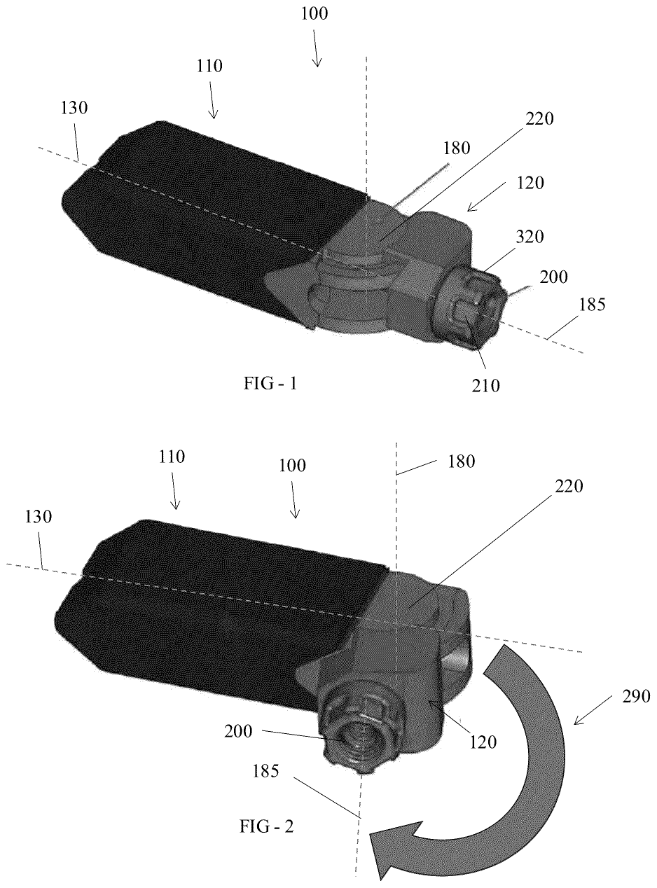

Example embodiments will now be described in more detail with respect to FIGS. 1-15. On FIGS. 1-15, similar reference numbers are used for representing similar elements. The following is a listing of reference numbers used on FIGS. 1-15 and their corresponding elements.
100 spacer
110 distal end
120 proximal end
130 distal end longitudinal axis
180 pivoting axis
185 proximal end longitudinal axis
200 drive nut interior threads
210 drive nut exterior notches
220 hinge
230 male threaded connection
240 wire rope subassembly
250 hollow threaded terminal
255 terminal exterior male threads
260 anchor terminal
264 passageway
270 wire rope
274 sleeve portion
276 enlarged head
280 terminal interior female threads
290 angle
300 inserter
310 nut driver
320 drive nut
330 first endplate
350 second endplate
360 sweeping interior void
364 sweeping interior edge
366 straight interior edge
370 expansion wedge
400 driver rotation
420 drive nut rotation
440 longitudinal translation of hollow threaded terminal
445 longitudinal translation of anchor terminal
450 plate expansion
500 distal ramped surface of first plate
520 distal ramped surface of second plate
550 first ramped surface of expansion wedge
570 second ramped surface of expansion wedge
600 first ramped surface of hinge
620 second ramped surface of hinge
650 proximal ramped surface of first plate
670 proximal ramped surface of second plate

With reference to FIG. 1, the disclosure provides various embodiments of an expandable spacer 100 having an adjustable height. The spacer is inserted between two adjacent bony surfaces to facilitate separation of the bones, and if desired, to promote the fusion of bony surfaces. Although intended to be useful with any adjacent bony surface in which fusion is desired, the spacer is advantageously applied to insertion between two adjacent vertebral bodies in any section of the spine, including the cervical, thoracic, lumbar, and sacral vertebral sections. More than one spacer 100 may be used within the body, for example between successive or separated vertebrae, or between adjacent vertebrae. The use of multiple spacers is particularly advantageous for patients whose back pain is not limited to a localized area, or for patients whose localized damage has progressed to other areas of the spine.

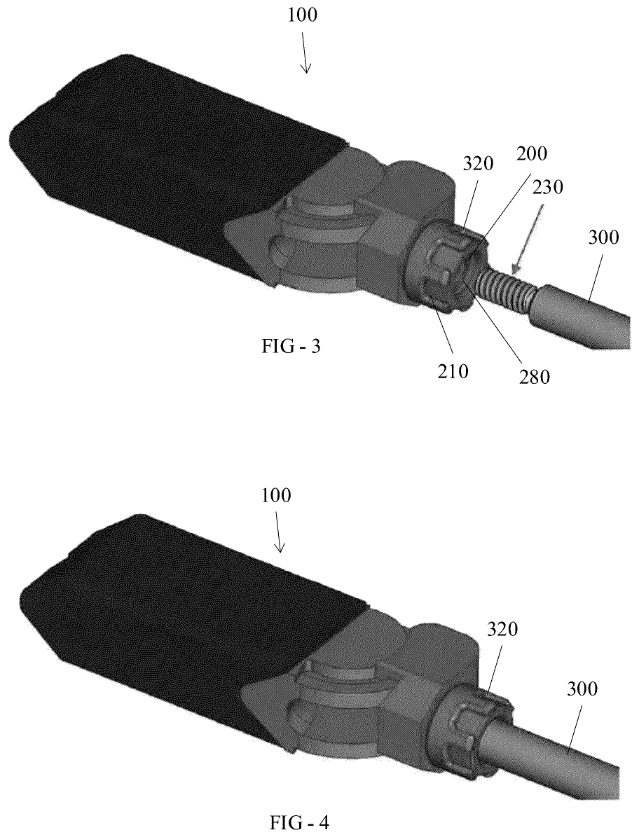

Embodiments of the spacer 100 may include a distal end 110 which may be inserted first into the body, and which can be tapered to facilitate insertion between body tissue, and a proximal end 120, to which a tool, such as an inserter 300 (e.g., shown on FIG. 3) or driver 310 (e.g., shown on FIG. 8) may be connected. The proximal end 120 has a proximal end longitudinal axis 185 while the distal end 110 has a distal end longitudinal axis 158. In this initial state, during the initial insertion into the body, the proximal end longitudinal axis 185 may be generally aligned with or parallel to the distal end longitudinal axis 158. A drive nut 320 may extend from the proximal end 120 and may contain interior female threads 200 as well as exterior notches 210.

With additional reference to FIG. 2, some embodiments of the spacer 100 include a hinge 220 which allows the proximal end longitudinal axis 185 to be angled relative to the distal end longitudinal axis 158 (in other words, the proximal end 120 has been pivoted relative to the distal end 110 about the pivoting axis 180 which is generally perpendicular to both the proximal end longitudinal axis 185 and distal end longitudinal axis 158). The angle between the proximal end longitudinal axis 185 and distal end longitudinal axis 158 may be referred to as angle 290, which could be any angle between zero and 90 degrees. Some embodiments of the spacer 100 can expand even when the distal end 110 and the proximal end 120 are in these positions (in other words once the proximal end longitudinal axis 185 has been angled 290 relative to the distal end longitudinal axis 158 to any angle between zero and 90).

FIG. 3 is a perspective view of an embodiment of the spacer 100 showing an initial step in the procedure where the spacer 100 is being attached to an inserter 300 using a male threaded connection 230 which is sized to engage with terminal interior female threads 280 of the hollow threaded terminal 250 (not visible in this Figure). The exterior notches 210 may not be used during this step in some embodiments.

FIG. 4 is a perspective view of an embodiment of the spacer 100 where the spacer 100 is being inserted into the body (e.g., not shown) using the inserter 300 which is generally an elongate member with male threaded connection 230 (e.g., shown on FIG. 3) on one end.

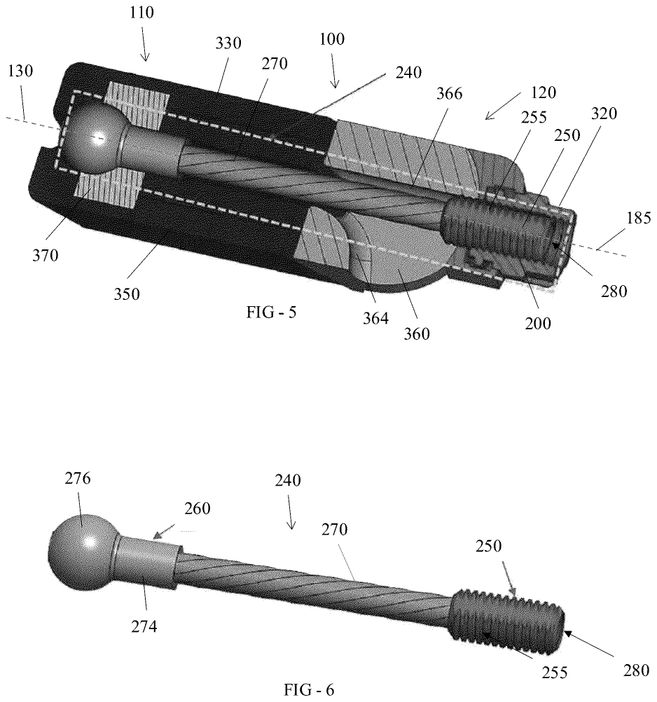

FIG. 5 is a section view of an embodiment of the spacer 100 of FIG. 1 taken vertically along the distal end longitudinal axis 158 and the proximal end longitudinal 185. A wire rope subassembly 240 may extend down a center of the spacer from the distal end 110 to the proximal end 120. As illustrated, the wire rope subassembly 240 may include a length of wire rope 270. A sweeping interior void 360 maybe positioned within the proximal end 120 to allow for the wire rope subassembly 240 to bend or deflect as the proximal end 120 pivots relative to the distal end 110. The sweeping interior void 360 may be defined on a first side by a straight interior edge 366 which may be generally parallel to the proximal end longitudinal axis 185. When the spacer 100 is in this position, the straight interior edge 366 may also be parallel to the wire rope assembly 240 and/or the wire rope 270. A second side of the sweeping interior void 360 may be defined by a sweeping interior edge 364 which may be positioned opposite the straight interior edge 366 with the sweeping interior void 360 defined in between these edges. The location of the sweeping interior edge 364 may represent the maximum amount of bend or deflection in the wire rope 270 (or maximum rotation of the proximal end 120 relative to the distal end 110).

The distal end 110 may include a first end plate 330 as well as a second end plate 350 with an expanding wedge 370 positioned in between the first and second end plates 330, 350.

The wire rope subassembly 240 may be connected at a first end with the expanding wedge 370 and at a second end with the drive nut 320. The wire rope assembly 240 may contain a length of wire rope 270 or flexible elongate member which is connected at a first end with a terminal anchor 260 and a second end with a hollow threaded terminal 250 having interior female threads 280 and exterior male threads 255. The drive nut interior threads 200 may engage with the terminal exterior male threads 255 such that the rotation of the drive nut 320 causes the hollow threaded terminal 250 to move longitudinally through the center of the drive nut 320 and away from the expansion wedge 370.

FIG. 6 is a perspective view of an embodiment of the wire rope assembly 240 having a length of wire rope 270 which is connected at a first end with a terminal anchor 260 and a second end with a hollow threaded terminal 250 having interior female threads 280 and exterior male threads 255. As illustrated, the terminal anchor 260 may include a sleeve portion 274 and an enlarged head 276 which extends distally from the sleeve portion 274. The wire rope 270 may attach to the sleeve portion 274, the enlarged head 276 or both. In the illustrated embodiment, the enlarged head 276 may have at least as partially rounded outer surface. In some embodiments, the enlarged head 276 may be a partial sphere.

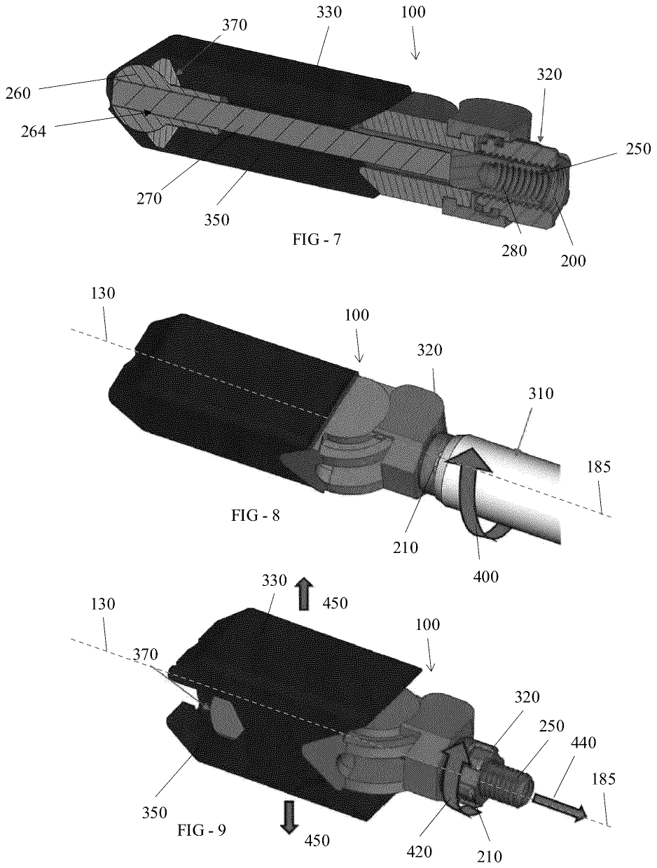

FIG. 7 is a perspective section view of an embodiment of the spacer 100 of FIG. 1 taken along the distal end longitudinal axis 158 and the proximal end longitudinal 185. The terminal anchor 260 may sit within a portion of the expanding wedge 370 while the hollow threaded terminal 250 may be engaged with the drive nut interior threads 200 such that rotation of the drive nut 320 causes the hollow threaded terminal 250 to translate longitudinally. The terminal anchor 260 may be attached to the wire rope 270 by any suitable means. As illustrated, the terminal anchor 260 may disposed over an end of the wire rope 270, for example, with the wire rope 270 extending through a through passage 264 in the terminal anchor 260. The distal end 110 may include a first end plate 330 as well as a second end plate 350 with an expanding wedge 370 positioned in between the first and second end plates 330, 350.

FIG. 8 is a perspective view of a driver 310 engaging with the exterior notches 210 of the drive nut 320 to cause rotation of the drive nut 320. The driver rotation 400 is shown generally as rotating about its central axis which may be generally aligned and/or parallel to axis 185 when the spacer 100 is in this position.

FIG. 9 is a perspective view of a driver 310 engaging with the drive nut 320 to cause rotation of the drive nut 320 where the driver 310 has been hidden to show the translation of the hollow threaded terminal 250 as well as the simultaneous expansion of the two end plates 330 and 350. As shown here, the first and second end plates 330 and 350 can be expanded when the longitudinal axes 158 and 185 are generally aligned or parallel, or when angled to one another (as shown in other Figures). In this aligned position, the driver 310 is also generally parallel to the longitudinal axes 158 and 185. The hollow threaded terminal 250 may translate longitudinally and towards the driver 310. In some embodiments, the hollow threaded terminal 250 is inside the drive nut 320 only during insertion into the body and is then outside of the drive nut 320 once the spacer has been expanded. The driver rotation 400 may cause engagement of the driver 310 with the exterior notches 210 in the drive nut 320 so that drive nut rotation 420 is generated which then generates the plate expansion 450.

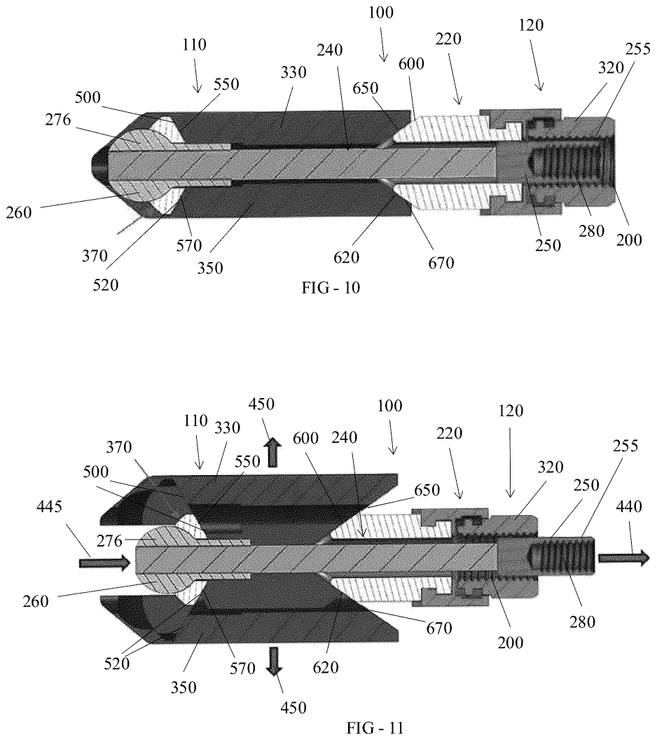

FIG. 10 is a section view of an embodiment of the spacer 100 of FIG. 1 taken along the distal end longitudinal axis 158 and the proximal end longitudinal 185 where the plates are in their initial state. The first end plate 330 may include a distal ramped surface 500 which corresponds to a first ramped surface 550 on the expansion wedge 370. Similarly, the second end plate 350 may contain a distal ramped surface 520 which corresponds to a second ramped surface 570 on the expansion wedge 370. A drive nut 320 may extend from the proximal end 120 and may contain interior female threads 200 (which engage with terminal exterior male threads 255 of the hollow threaded terminal 250) as well as exterior notches 210.

The enlarged head 276 may be positioned within the expansion wedge 370 so that linear movement of the enlarged head 276 may be transferred to the expansion wedge 370. The enlarged head 276 is preferably locked or contained within the expansion wedge 370 such that the two components move together, or in other words the enlarged head 276 should be sized larger than the receiving portion of the expansion wedge 370 such that the enlarged head 276 could never be pulled through the expansion wedge 370.

The first end plate 330 may include a proximal ramped surface 650 which corresponds to a first ramped surface 600 of the hinge 220. Similarly, the second end plate 350 may contain a proximal ramped surface 670 which corresponds to the second ramped surface 620 of the hinge 220. The distal ramped surface 500 of the first place 330 as well as the distal ramped surface 520 of the second plate 350 may both be positioned near a distal end 110 of the device and each may be facing the anchor terminal 260. The proximal ramped surface 650 of the first plate 330 and the proximal ramped surface 670 of the second plate 350 may both be positioned near a proximal end 120 of the device and each may be facing the hollow threaded terminal 250.

The first ramped surface 550 of the expansion wedge 370 and the second ramped surface 570 of the expansion wedge may be facing the proximal end 120 of the spacer 100. The first ramped surface 550 of the expansion wedge 370 and the second ramped surface 570 of the expansion wedge may also be facing the hollow threaded terminal 250 and drive nut 320. The first ramped surface 600 of the hinge 220 and the second ramped surface 620 of the hinge 220 may be facing the distal end 110 of the spacer 100. The first ramped surface 600 of the hinge 220 and the second ramped surface 620 of the hinge 220 may also be facing the anchor terminal 370.

The wire rope subassembly 240 may be generally straight in this position and thus may not have a bend or curve in the wire rope subassembly 240, as least during insertion and placement of the spacer 100.

FIG. 11 is a section view of an embodiment of the spacer 100 of FIG. 9 taken along the distal end longitudinal axis 158 and the proximal end longitudinal 185 where the plates are expanding 450 and the hollow threaded terminal 250 is translating through the drive nut 320. In order to cause the plate expansion 450 (first end plate 330 and second end plate 350 move away from one another), the wire rope subassembly 240 may translate longitudinally causing the anchor terminal 260 to also translate the expanding wedge 370 to also translate which causes the ramped surfaces 550 and 570 of the expanding wedge 370 to slide along the ramped surfaces 500 and 520 of the first end plate and second end plate. As the ramped surfaces slide along one another, the first end plate 330 may move away from the second end plate 350.

As the drive nut 320 rotates relative to the proximal end 120, the wire rope subassembly translates, including the hollow threaded terminal 250 which may have a longitudinal translation 440. Simultaneously, the anchor terminal 260 should also have a longitudinal translation 445 which forces the ramped surfaces 550 and 570 of the expanding wedge 370 to slide along the ramped surfaces 500 and 520 thus causing the first end plate 330 and second end plate 350 to further separate to any desired position or distance apart.

The longitudinal translation 445 of the anchor terminal 260 may also force the proximal ramped surface 650 to slide along the first ramped surface 600 of the hinge 200 and simultaneously force the proximal ramped surface 670 to slide along the second ramped surface 620 of the hinge 220. As shown in the figure, the sliding of proximal ramped surface 650 along the first ramped surface 600 of the hinge 220 in addition to the sliding of proximal ramped surface 670 along the second ramped surface 620 of the hinge 220 can be used to add additional forces to the plate expansion 450.

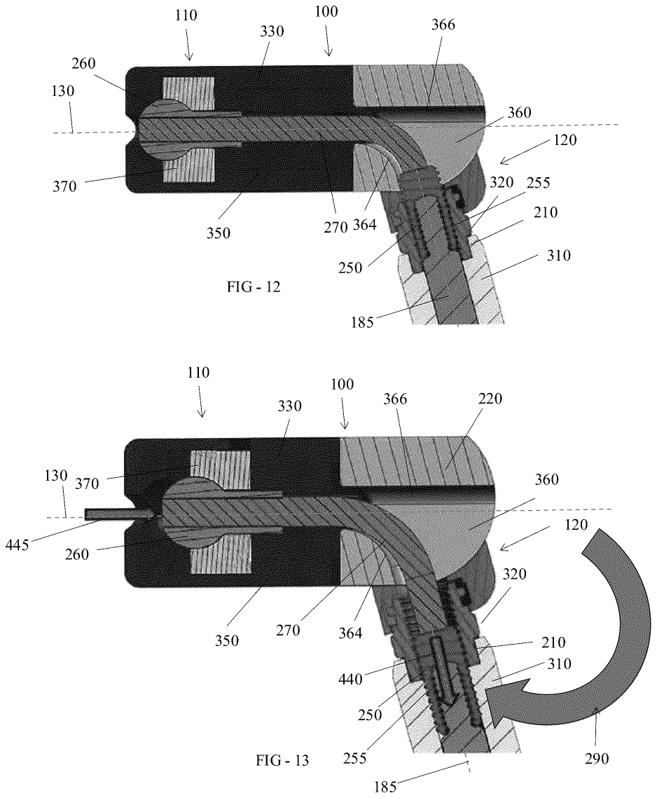

FIG. 12 is a section view of an embodiment of the spacer where the proximal end 120 has articulated or pivoted relative to the distal end 110 and the driver 310 is engaged with the drive nut 320. To arrive at this position, the wire rope 270 may bend or rotate through the sweeping interior void 360. While the initial position of the spacer 100 during insertion (e.g., see FIG. 5) shows the wire rope 270 substantially parallel to and against the straight interior edge 366, the sweeping interior void 360 allows the wire rope 270 to bend and fill any location within the sweeping interior void 360 between the straight interior edge 366 and the sweeping interior edge 364, or along either the straight interior edge 366 or the sweeping interior edge 364. In the position shown, at least a portion of the wire rope 270 may be positioned along the sweeping interior edge 364. Also in this position, a portion of the wire rope 270 near the anchor terminal 260 is shown generally parallel to the distal end longitudinal axis 130. However, another portion of the wire rope 270 that is near the hollow threaded terminal 250 is shown angled relative to the distal end longitudinal axis 130.

The driver 310 may engage with the exterior notches 210 of the drive nut 320. To begin expanding the first end plate 330 away from the second end plate 350, the driver 310 may be rotated about its axis which is generally aligned and parallel to the proximal end longitudinal axis 185. The longitudinal axis 130 is generally not aligned with the longitudinal axis 185 when the spacer 100 is in this articulated position. The anchor terminal 260 may be positioned against the expansion wedge 370 and will generally translate longitudinally along the longitudinal axis 130 while the hollow threaded terminal 250 translates longitudinally along the longitudinal axis 185.

FIG. 13 is a section view of an embodiment of the spacer where the proximal end 120 has articulated or pivoted relative to the distal end 110 and the rotation of the drive nut 320 is causing the terminal anchor 260 and the hollow threaded terminal 250 to translate along their respective longitudinal axes 158 and 185. The distal end 110 may include a first end plate 330 as well as a second end plate 350 with an expanding wedge 370 positioned in between the first and second end plates 330, 350.

The longitudinal translation 440 of the hollow threaded terminal 250 may be transferred by the wire rope 270 to the anchor terminal 260 in the form of longitudinal translation 445. The hinge 220 may allow the proximal end longitudinal axis 185 to be angled relative to the distal end longitudinal axis 158 (in other words, the proximal end 120 has been pivoted relative to the distal end 110 about the pivoting axis 180 which is generally perpendicular to both the proximal end longitudinal axis 185 and distal end longitudinal axis 158). The angle between the proximal end longitudinal axis 185 and distal end longitudinal axis 158 may be referred to as angle 290, which could be any angle between zero and 90 degrees. The longitudinal axis 130 is generally not aligned with the longitudinal axis 185 when the spacer 100 is in this articulated position. The anchor terminal 260 may be positioned against the expansion wedge 370 and will generally translate longitudinally along the longitudinal axis 130 while the hollow threaded terminal 250 translates longitudinally along the longitudinal axis 185.

The portion of the wire rope 270 that is near the anchor terminal 260 may be generally straight and parallel to the distal end longitudinal axis 130. The portion of the wire rope 270 that is near the hollow threaded terminal 250 may also be generally straight and parallel to the proximal end longitudinal axis 185. Thus, the angle 290 between the proximal end longitudinal axis 185 and the distal end longitudinal axis 130 may also represent the angle 290 between the portion of the wire rope 270 that is near the hollow threaded terminal 250 and the portion of the wire rope 270 that is near the hollow threaded terminal 250. A bend or curve in the wire rope 270 may be positioned between these two portions of the wire rope 270 and said bend or curve may be positioned along the sweeping interior edge 364 when the maximum value for the angle 290 has been reached. At other positions between the one shown here and the initial position shown in FIG. 5, the wire rope 270 and the bend or curve in the middle may occupy any position within the sweeping void 360 which provides the open space for the wire rope 270 to travel as the proximal end 120 articulates or pivots relative to the distal end 110.

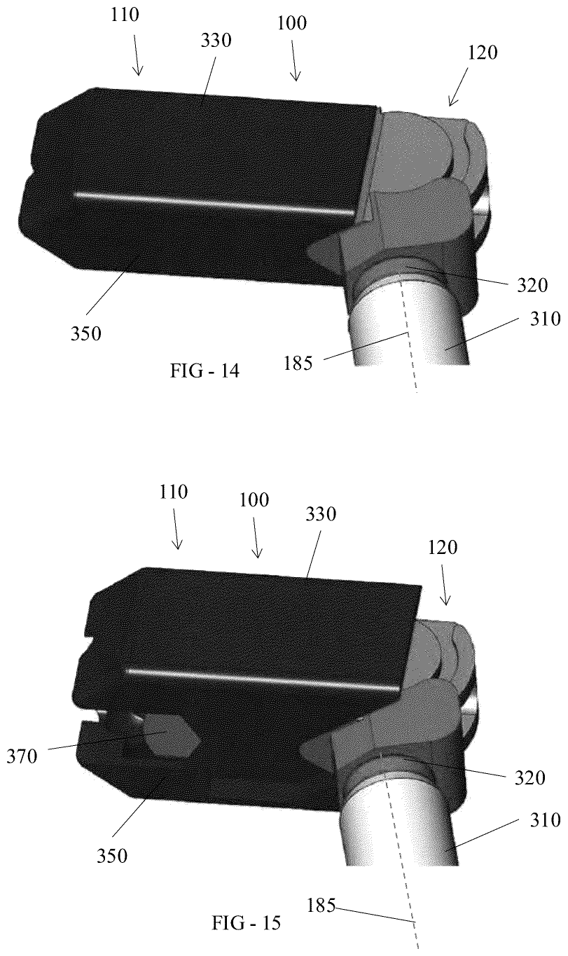

FIG. 14 is a perspective view of an embodiment of the spacer 100 where the proximal end 120 has articulated or pivoted relative to the distal end 110 and the driver 310 is engaged with the drive nut 320. To begin expanding the first end plate 330 away from the second end plate 350, the driver 310 may be rotated about its axis which is generally aligned and parallel to the proximal end longitudinal axis 185.

FIG. 15 is a section view of an embodiment of the spacer 100 where the proximal end 120 has articulated or pivoted relative to the distal end 110 and the end plates 330 and 350 have been expanded while the spacer 100 has been articulated to the desired angle or position. The driver 310 may remain aligned with the longitudinal axis 185 of the proximal end 120 while also remaining engaged with the drive nut 320 to continue expanding the first end plate 330 away from the second end plate 350 until reaching the desired expansion distance. The expansion wedge 350, as shown above in detail, is positioned between the first end plate 330 and second end plate 350 and engages with both plates 330 and 350 to cause the plates to expand or translate away from each other, as shown and described herein.

It should be noted that wire rope 270 is not specifically required for these embodiments as any flexible member with tensile strength would also work in various embodiments, including a plastic or flexible metal or composite material.

The spacer 100 and methods for its insertion can be used in a treatment protocol for any of a wide variety of conditions in a patient involving diseased or damaged bony structures. The patient can be a human being. Additionally, it is contemplated that the spacer 100 may be useful in veterinary science for any animal having adjacent bony structures to be fused. The spacer 100 can collapse, for example, to approximately one half of its expanded size. When in this collapsed configuration, the spacer can be inserted into a space through a small incision and narrow pathways, using appropriate minimally-invasive techniques, and can be positioned within the space between adjacent bones, and then expanded to a desired therapeutic height. The incision may be short, for example about one inch in length, which is smaller than the spacer in an expanded configuration. If the desired position and/or expansion are not achieved, the spacer can be collapsed, repositioned, and re-expanded in situ.

Although the spacer 100 is exemplified herein for use in the spine, the spacer is contemplated for fusion of any bony structures. While the spacers 100 are described herein using several varying embodiments, the spacers are not limited to these embodiments. An element of one embodiment may be used in another embodiment, or an embodiment may not include all described elements.

With respect to all embodiments, in accordance with the disclosure, during insertion of intervertebral spacers 100 from a posterior approach, there is a need to avoid damaging nerve roots. A prior art spacer dimensioned to separate bones can block a view of nerve roots as it is inserted, and due to its large size, poses a greater risk of contacting nerve roots during insertion into the body. As a result, the medical practitioner must more often retract nerve roots, with attendant danger of tissue damage. Spacers 100 in accordance with example embodiments form a smaller dimension during insertion, relative to a final dimension for spacing bones. Accordingly, nerve roots can be visualized and avoided during insertion, and nerve root manipulation can be avoided or minimized.

As spacers 100 in accordance with example embodiments can be articulated during insertion, they can be inserted between bones by being passed through a minimally invasive entry, for example through an incision approximating the smallest collapsed dimension, for example transverse to a longitudinal dimension extending between distal and proximal ends 110 and 120 respectively. This enables exceptional anterior placement without impaction, as well as facilitating insertion from other approaches. Spacers 100 in accordance with example embodiments further develop a good bone contact area, as a spacer with a larger footprint may be inserted through a reduced size incision, due to the overall dimensions of the spacer being reduced during insertion.

Spacers 100 in accordance with example embodiments enable a continuous expansion and retraction over a range of displacements according to predetermined dimensions of a specific spacer design. This provides the ability to distract vertebral bodies or other bones to a desired height or separation. First and second endplates 330 and 350 can be shaped to form planes or surfaces which converge relative to each, to provide for proper lordosis, and can be provided with openings through which bone may grow, and into which bone graft material may be placed. Spacers 100 of the disclosure may be used to distract, or force bones of a joint apart, or may be used to maintain a separation of bones created by other means, for example by a retractor. First and second endplates 330 and 350 may additionally be curved to conform to the surface of body tissue, for example the surface of cortical bone, of the vertebra to be contacted, for improved fixation and load bearing. Spacers 100 of the disclosure may be further secured in connection with the body by passage of elongated fasteners through one or both of the first and second endplates 330 and 350.

Spacers 100 in accordance with examples embodiments may be fabricated using any biocompatible materials known or hereinafter discovered, having sufficient strength, flexibility, resiliency, and durability for the patient, and for the term during which the device is to remain implanted in the body. Examples include but are not limited to metal, such as, for example titanium and chromium alloys; stainless steel, polymers, including for example, PEEK or high molecular weight polyethylene (HMWPE); and ceramics. There are many other biocompatible materials which may be used, including other plastics and metals, as well as fabrication using living or preserved tissue, including autograft, allograft, and xenograft material.

Portions or all of the spacer 100 may be radiopaque or radiolucent, or materials having such properties may be added or incorporated into the spacer to improve imaging of the device during and after insertion.

Spacers 100 may be formed using titanium, or a cobalt-chrome-molybdenum alloy, Co—Cr—Mo, for example as specified in ASTM F1537 (and ISO 5832-12). The smooth surfaces may be plasma sprayed with commercially pure titanium, as specified in ASTM F1580, F1978, F1147 and C-633 (and ISO 5832-2). Alternatively, part or all of spacers 100 may be formed with a polymer, for example ultra-high molecular weight polyethylene, UHMWPE, for example as specified in ASTM F648 (and ISO 5834-2). In one embodiment, PEEK-OPTIMA (a trademark of Invibio Ltd Corp, United Kingdom) may be used for one or more components of the spacers of the disclosure. For example, polymeric portions can be formed with PEEK-OPTIMA, which is radiolucent, whereby bony ingrowth may be observed. Other polymeric materials with suitable flexibility, durability, and biocompatibility may also be used.

In accordance with various embodiments shown herein, spacers 100 of various sizes may be provided to best fit the anatomy of the patient. Components of matching or divergent sizes may be assembled during the insertion procedure by a medical practitioner as best meets the therapeutic needs of the patient, the assembly inserted within the body using an insertion tool. Some embodiments may also be provided with an overall angular geometry, for example an angular mating disposition of the first and second endplates 330 and 350, to provide for a natural lordosis, or a corrective lordosis, for example of from 0° to 12° for a cervical application, although much different values may be advantageous for other joints. Lordotic angles may also be formed by shaping one or both endplates 330 and 350 to have relatively non-coplanar surfaces.

Expanded spacer heights, for use in the cervical vertebrae for example, may typically range from 7 mm to 12 mm, but may be larger or smaller, including as small as 5 mm, and as large as 16 mm, although the size is dependent on the patient, and the joint into which an embodiment of the spacer 100 is to be inserted. Spacers 100 may be inserted within any level of the spine, and may also be inserted into other joints of the body, including joints of the hand, wrist, elbow, shoulder, hip, knee, ankle, or foot.

In accordance with some embodiments, a single spacer 100 may be used, to provide stabilization for a weakened joint or joint portion. Alternatively, a combination of two, three, or more of any of spacer 100 may be used, at a single joint level, or in multiple joints. Moreover, spacers of the disclosure may be combined with other stabilizing means.

Additionally, spacers 100 in accordance with example embodiments may be fabricated using material that biodegrades in the body during a therapeutically advantageous time interval, for example after sufficient bone ingrowth has taken place. Further, spacers of the disclosure are advantageously provided with smooth and or rounded exterior surfaces, which reduce a potential for deleterious mechanical effects on neighboring tissues.

Any surface or component of a spacer of the disclosure may be coated with or impregnated with therapeutic agents, including bone growth, healing, antimicrobial, or drug materials, which may be released at a therapeutic rate, using methods known to those skilled in the art.

Devices of the disclosure provide for adjacent vertebrae to be supported during flexion/extension, lateral bending, and axial rotation. In one embodiment, spacer 100 is indicated for spinal arthroplasty in treating skeletally mature patients with degenerative disc disease, primary or recurrent disc herniation, spinal stenosis, or spondylosis in the lumbosacral spine (LI-SI). Degenerative disc disease is advantageously defined as discogenic back pain with degeneration of the disc confirmed by patient history and radiographic studies, with or without leg (radicular) pain. Patients are advantageously treated, for example, who may have spondylolisthesis up to Grade 1 at the involved level. The surgery position spacer 100 may be performed through an Anterior, Anterolateral, Posterolateral, Lateral, or any other approach.

In a typical embodiment, spacers 100 in accordance with example embodiments have an uncompressed height, before insertion, of 7 to 13 mm, and may advantageously be provided in cross-sections of 10×26 mm, 12×31 mm and 12×36 mm, with 4, 8, 12, or 16 degree lordotic angles, although these are only representative sizes, and substantially smaller or larger sizes can be therapeutically beneficial. In one embodiment, spacers 100 in accordance with example embodiments may be sized to be inserted using an MIS approach, for example using a reduced incision size, for example less than about 5 cm, and advantageously less than about 2.5 cm, with fewer and shorter cuts through body tissue. Spacer 100 may advantageously be used in combination with other known or hereinafter developed forms of stabilization or fixation, including for example rods and plates.

Spacers 100 in accordance with example embodiments can be inserted into the body, advantageously in a collapsed or non-expanded configuration, through a transforaminal approach, and can articulate in attachment to an inserter tool (not shown), for example for anterior placement. Once placement is achieved, the spacer 100 is capable of expanding for disc height restoration.

All references cited herein are expressly incorporated by reference in their entirety. There are many different features to the present invention and it is contemplated that these features may be used together or separately. Unless mention was made above to the contrary, it should be noted that all of the accompanying drawings are not to scale. Thus, the invention should not be limited to any particular combination of features or to a particular application of the invention. Further, it should be understood that variations and modifications within the spirit and scope of the invention might occur to those skilled in the art to which the invention pertains. Accordingly, all expedient modifications readily attainable by one versed in the art from the disclosure set forth herein that are within the scope and spirit of the present invention are to be included as further embodiments of the present invention.

1. A spacer for positioning between first and second bones of adjacent vertebral bodies, the spacer comprising:
a distal end having a longitudinal axis and comprising:
a first endplate configured to engage the first bone;
a second endplate configured to engage the second bone; and
an expansion wedge positioned between the first endplate and second endplate;
a proximal end connected to the distal end and having a proximal end longitudinal axis;
a drive nut extending from the proximal end; and
a wire rope assembly extending down a center of the spacer and connected at a first end with the expansion wedge and at a second end with the drive nut.

2. The spacer of claim 1 wherein:
the wire rope assembly comprises:
a hollow threaded terminal;
an anchor terminal; and
a section of wire rope connecting between the hollow threaded terminal and the anchor terminal.

3. The spacer of claim 2 wherein:
the hollow threaded terminal comprises:
interior female threads; and
exterior male threads.

4. The spacer of claim 1 further comprising:
a hinge connecting between the proximal end and the distal end;
a proximal ramped surface on the first plate which engages with a first ramped surface on the hinge; and
a proximal ramped surface on the second plate which engages with a second ramped surface on the hinge.

5. The spacer of claim 1 wherein:
the distal end is hingedly fastened to the proximal end such that the distal end can pivot relative to the proximal end.

6. The spacer of claim 1 wherein:
the distal end pivots about a pivoting axis after the proximal end is positioned between the first bone and second bone.

7. The spacer of claim 1 wherein:
rotation of the drive nut causes the first endplate to move away from the second endplate while the proximal end longitudinal axis is angled relative to the distal end longitudinal axis.

8. The spacer of claim 1 wherein:
rotation of the drive nut causes the spacer to expand after the proximal end has been pivoted relative to the distal end.

9. The spacer of claim 1 wherein:
rotation of the drive nut causes the wire rope assembly to translate along the longitudinal axis of the distal end and the proximal end.

10. The spacer of claim 1 wherein:
translation of the wire rope assembly causes an increase in a height of the spacer.

11. A spacer for positioning between first and second bones of adjacent vertebral bodies, the spacer comprising:
a distal end comprising:
a first endplate configured to engage the first bone;
a second endplate configured to engage the second bone; and
an expansion wedge positioned between the first endplate and second endplate;
a hinge connected to a proximal end;
a drive nut extending from the hinge;
an anchor terminal positioned inside the expansion wedge;
a hollow threaded terminal positioned inside the drive nut; and
a flexible member extending between the anchor terminal of the expansion wedge and hollow threaded terminal of the drive nut.

12. The spacer of claim 11 wherein:
the hollow threaded terminal comprises:
interior female threads; and
exterior male threads.

13. The spacer of claim 11 wherein:
the hinge allows the proximal end to pivot relative to the distal end after the distal end has engaged with the first bone and second bone.

14. The spacer of claim 11 wherein:
rotation of the drive nut engages with the hollow threaded terminal to translate the anchor terminal through the expansion wedge.

15. The spacer of claim 14 wherein:
translation of the anchor terminal through the expansion wedge causes the first endplate and second endplate to expand.

16. The spacer of claim 12 wherein:
the interior female threads of the hollow threaded terminal engage with an inserter.

17. The spacer of claim 11 further comprising:
a distal ramped surface on the first plate which engages with a first ramped surface on the expansion wedge; and
a distal ramped surface on the second plate which engages with a second ramped surface on the expansion wedge.

18. The spacer of claim 11 wherein:
a longitudinal axis of the distal end and the longitudinal axis of the proximal end are angled to each other while the spacer expands.

19. A spacer for positioning between first and second bones of adjacent vertebral bodies, the spacer comprising:
a distal end comprising:
a first endplate configured to be positioned between the first and second bones;
a second endplate configured to be positioned between the first and second bones; and
an expansion wedge positioned between the first endplate and second endplate;
a hinge connected to a proximal end;
a sweeping void placed inside the hinge;
a drive nut extending from the hinge;
an anchor terminal positioned inside the expansion wedge;
a hollow threaded terminal positioned inside the drive nut and comprising:
interior female threads sized to engage with an inserter; and
exterior male threads sized to engage with female threads inside the drive nut; and
a wire rope member extending between the anchor terminal of the expansion wedge and hollow threaded terminal of the drive nut and permitted to bend within the sweeping void.

20. The spacer of claim 19 further comprising:
a distal ramped surface on the first plate which engages with a first ramped surface on the expansion wedge;
a distal ramped surface on the second plate which engages with a second ramped surface on the expansion wedge;
a proximal ramped surface on the first plate which engages with a first ramped surface on the hinge; and
a proximal ramped surface on the second plate which engages with a second ramped surface on the hinge.
